(19) United States
(12) Patent Application Publication
(10) Pub. No.: US 2026/0317734 A1
(43) Pub. Date: Oct. 15, 2026

(54) Systems, Compositions, and Methods to Control the Integrated Stress Response

(71) Applicant: The Regents of the University of California, Oakland, CA (US)

(72) Inventors: Maxwell Wilson, Santa Barbara, CA (US); Taivan Batjargal, Goleta, CA (US); Diego Acosta-Alvear, Belmont, CA (US)

(73) Assignee: The Regents of the University of California, Oakland, CA (US)

(21) Appl. No.: 19/485,231

(22) PCT Filed: May 17, 2024
(86) PCT No.: PCT/US2024/029956

§ 371 Date: Nov. 17, 2025

Related U.S. Application Data
(60) Provisional application No. 63/502,898, filed on May 17, 2023.

(51) Int. Cl. C12N 9/12; C12N 13/00; C12N 15/52; C12N 15/62; C12N 15/86; G01N 33/68

(52) U.S. Cl. CPC C12N 9/12; C12N 13/00; C12N 15/52; C12N 15/62; C12N 15/86; C12Y 207/11001; G01N 33/68; C12N 2830/34

(57) ABSTRACT

Many embodiments are directed to systems, compositions, and methods to control the ISR. Numerous embodiments utilize optogenetic control to control the ISR. Optogenetic control can be provided by an engineered ISR kinase that can dynamically stimulate the ISR pathway. Using optogenetic control, many embodiments enable the selective activation of a single kinase of the ISR pathway without inducing real molecular damage, greatly reducing the combinatorial complexity of the signals that are turned on by real stressors. By changing the intensity of light stimulation, various embodiments can tune the fraction of optogenetic proteins in the activated state, thereby precisely altering the perceived intensity. Additionally, precisely defined input dynamics can be delivered by toggling lights between on and off positions. Many embodiments are scalable and/or capable of high-throughput delivery, allowing for precise dissections of the ISR pathway.

### CROSS REFERENCE TO RELATED APPLICATIONS

This application claims priority to U.S. Provisional Application Ser. No. 63/502,898, entitled “Systems, Compositions, and Methods to Control the Integrated Stress Response,” filed May 17, 2023, the disclosure of which is hereby incorporated by reference in its entirety.

### FIELD OF THE INVENTION

The present invention relates to engineered proteins that an optical activating domain and an effector domain capable of effecting an integrated stress response without an external stressor.

### BACKGROUND

The Integrated Stress Response (ISR) is a conserved signaling network that allows cells to detect and react to cell-intrinsic and environmental stresses including nutritional deficits, mitochondrial dysfunction, redox imbalances, viral infections, harmful RNA conformers, and protein-folding perturbations in the endoplasmic reticulum (ER). Because of its fundamental nature, it is not surprising that dysregulation of the ISR has been implicated in the pathogenesis of our most widespread diseases, including diabetes, cancer, and neurodegeneration. The mammalian ISR is governed by four stress sensor kinases—PERK, PKR, HRI, and GCN2—all of which actuate signaling by phosphorylating a single serine of the α subunit of the eukaryotic translation initiation factor eIF2 (eIF2α), a heterotrimeric GTPase required for translation initiation. Phosphorylated eIF2α (p-eIF2α) is a potent competitive inhibitor of its guanine nucleotide exchange factor eIF2B thereby leading to a global shutdown of protein synthesis. Concomitantly, phosphorylation of eIF2α promotes the translation of select mRNAs, including those encoding the ISR effector transcription factors ATF4, CHOP, as well as GADD34, the regulatory subunit of protein phosphatase 1 that mediates negative feedback on the system through dephosphorylation of eIF2α to subdue ISR signals. While the molecular structure and circuitry of the ISR in health and disease have been extensively studied, major questions remain about how the ISR encodes information at the systems level, such as:
How does the intensity and duration of a stress determine the cell's response?
On what timescales and with what subnetworks does the ISR determine cell fate?
How do past stresses influence cellular resilience?

These questions are difficult to address with classical ISR-inducing agents such as chemicals (e.g., the ER calcium reuptake inhibitor thapsigargin and sodium arsenite), viral infection or synthetic RNAs (e.g., poly-I:C), and physical stimuli such as heat-shock, because all of these stressors introduce pleiotropic changes coupled to molecular damage that conflate bona fide changes that result from the cell's response to stress with changes resulting from failing cellular machinery. In addition, the dose-response relationship between real stressors and pathway activation is non-trivial because of the cascading failures that non-linearly alter cellular stress levels. Last, damage induced by stressors is not immediately reversible. Instead, it must be repaired by the cell, constraining the investigation of how the ISR decodes dynamic inputs. Because of these limitations, there is a need in the art for a methodology to dissect the ISR in a more precise manner.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In some aspects, the techniques described herein relate to an engineered protein including a protein kinase R (PKR), where at least one double-stranded RNA binding domain is replaced with a photo-switchable variant of Cryptomchrome2 (Cry2).

In some aspects, the techniques described herein relate to an engineered protein, where at least two double-stranded RNA binding domains are replaced with a photo-switchable variant of Cry2.

In some aspects, the techniques described herein relate to an engineered protein, where the photo-switchable variant of Cry2 contains an E490G mutation.

In some aspects, the techniques described herein relate to an engineered protein, where the photo-switchable variant of Cry2 is selected from: Cry2Olig, Cry2OClust, Cry2ODrop.

In some aspects, the techniques described herein relate to a nucleic acid encoding an above-described protein.

In some aspects, the techniques described herein relate to a cell containing an above-described composition.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1A illustrates a schematic of a construct encoding an optogenetic ISR effector comprising an effecting domain derived from a naturally occurring ISR effector and a Cry2 variant in accordance with various embodiments.

FIG. 1B illustrates a schematic representation of an optogenetic ISR effector induction of the ISR via light application in accordance with various embodiments.

FIG. 2 illustrates a method for dissecting a biochemical pathway in accordance with various embodiments.

FIG. 3A illustrates a schematic of a construct encoding opto-PKR. A Cry2Olig domain is fused to human PKR at residue 170 to 551. dRBM1/2, double-stranded RNA binding motifs in accordance with various embodiments.

FIG. 3B illustrates a schematic representation of opto-PKR-driven induction of the ISR. The light-responsive Cry2 domain reversibly oligomerizes leading to clustering and trans-autophosphorylation of opto-PKR (blue light) or shutdown of the response (no light) in accordance with various embodiments.

FIG. 3C illustrates live cell imaging of opto-PKR in H4 cells under constant blue light application in accordance with various embodiments.

FIG. 3D illustrates western blot of puromycin incorporation into nascent peptides under blue light conditions in cells bearing opto-PKR compared to wild-type in accordance with various embodiments.

FIG. 3E illustrates immunofluorescence analysis of the SG marker G3BP1 in accordance with various embodiments.

FIGS. 4A-4D illustrate the ISR has a biphasic transcriptional response to virtual stress in accordance with various embodiments, where FIG. 4A illustrates an experimental design to investigate transcriptomic dynamics of opto-PKR-induced ISR over time, H4-cells bearing opto-PKR were illuminated using the optoPlate device to collect 7 samples across 720 mins. FIG. 4B illustrates a plot of singular values from E matrix of singular value decomposition on a relative scale. FIG. 4C illustrates a plot of the top 500 and bottom 500 genes from each U vector associated with the most prominent two dynamic modes, σ1 and σ2. Each blue line represents a distinct gene from our z-scored RNA-seq data, the red line is the mean of 500 plotted genes over time. And, FIG. 4D illustrates a centroid-clustered heatmap of the global transcriptome dynamics over 12 hours. Rooted tree diagram of gene clustering is color coded to indicate 4 dynamic modes.

FIGS. 5A-5J illustrate that ISR transcription factors in different ISR signaling modes respond proportionally with fixed phases in accordance with various embodiments, where FIG. 5A provides a schematic of ISR information flow through activation of opto-PKR. FIG. 5B provides an experimental design to query responses to varying illumination intensity over time. Blue light intensity was varied from 0.2-7.5 mcd using the optoPlate device over the period of 600 mins. Cells were fixed and immunostained for the indicated markers, imaged and immunofluorescence data was processed with an automated pipeline. FIGS. 5C, 5E, and 5G illustrate representative immunofluorescence images of H4 cells bearing opto-PKR under the light exposures outlined, varying combinations of intensity and duration, stained for p-eIF2α (FIG. 5C), ATF4 (FIG. 5E) and CHOP (FIG. 5G), respectively. n>150. Scale bar=10 μm. FIGS. 5D, 5F, and 5H provide quantification of immunofluorescence data in FIGS. 5C, 5E, and 5G. Each line corresponds to a specific illumination intensity over time. Error bars: SEM. FIG. 5I provides a representative image of light-based gradient activation of opto-PKR in H4 cells immunostained for CHOP. The light gradient was applied using a digital micro-mirror device (DMD) and continuous illumination. Scale bar=100 μm. And, FIG. 5J provides a quantification of nuclear CHOP signal upon gradient light stimulation. Each point corresponds to the mean nuclear immunofluorescence intensity of all nuclei positioned within one of hundred equally divided horizontal space regions. Each line is a best fit adhering to a quadratic formula: f(x)=ax\hat{ }2+1. n>2900.

FIGS. 6A-6F provide a simple ODE model of the ISR captures core responses and predicts stress memory in accordance with various embodiments. FIG. 6A illustrates a schematic representation of interactions simulated in our model using equations representing opto-PKR, p-opto-PKR, eIF2α, p-eIF2α and GADD3-PP1 complex, segregated to three sub-modules of the ISR. FIG. 6B provides a best fit of simulated data to the cooperative ODE model to experimental data for p-eIF2α. The line is simulated data, and the points are experimentally measured data. FIG. 6C provides simulations showing changes in p-eIF2α levels in response to decreasing illumination intensity. FIG. 6D Simulated response of all state variables of our cooperative model after fitting to experimental data. FIG. 6E provides a demonstration of inputs used to probe system hysteresis. To investigate the effect of past stress inputs to present stress inputs, we simulated multiple increasing ratios of stress input to recovery in the same period, before applying an identical challenge to all simulations. FIG. 6F provides a simulated stress memory through modulation of input and recovery ratios. Normalized levels of key state variables over time: p-opto-PKR (left), p-eIF2α (mid), GADD34-PP1 (right). Challenge occurs at 10 hours for all simulations.

FIGS. 7A-7D illustrate input dynamics shape the stress memory landscape in accordance with various embodiments, where FIG. 7A illustrates an experimental scheme used to map the stress memory landscape. Cells were conditioned by varying stress input and recovery durations, before applying an identical challenge to query present response. Conditioning inputs were applied to the cooperative model and to cells for comparison. Cells were illuminated with the optoPlate device, fixed, immunostained, and quantified for p-eIF2α (similarly to FIG. 5B). FIG. 7B provides a heatmap of simulated stress memory landscape for p-eIF2α in response to 10 min challenge. Total duration of conditioning varies. FIG. 7C provides an interpolated heatmap of experimental stress memory landscape for p-eIF2α in response to 10 min challenge. Experimental data quantified as normalized mean intensity of cytoplasmic immunofluorescence signal. Each dot represents a sample point. n>375 single cells for each point. FIG. 7D Representative immunofluorescence images immunostained for p-eIF2α after conditioning and challenge. Controls: naïve=no illumination, +10 min=only challenge (no conditioning). Scale bar=10 μm.

FIGS. 8A-8C provide a construct design of opto-IRE1 and experimental data of its use in accordance with various embodiments. FIG. 8A provides a schematic of a construct encoding opto-IRE1. FIG. 8B provides live imaging of opto-IRE1 in HEK293T cells under pulsed blue light application. FIG. 8C provides data showing that expression of XBP1 and CHOP is induced upon constant blue light application in HEK293T stably expressing opto-IRE1.

FIGS. 9A-9B provide a construct design of opto-PERK and experimental data of its use in accordance with various embodiments. FIG. 9A provides a schematic of a construct encoding opto-PERK. FIG. 8B provides live imaging of opto-PERK in HEK293T cells under pulsed blue light application.

### DETAILED DESCRIPTION

The embodiments of the invention described herein are not intended to be exhaustive or to limit the invention to precise forms disclosed. Rather, the embodiments selected for description have been chosen to enable one skilled in the art to practice the invention.

As noted previously, studying the integrative stress response (ISR) has numerous challenges. In ideal approach to dissect this control would involve the following goals:
It would isolate a single stress sensor and activate it virtually to avoid conflation of inputs from parallel pathways and molecular damage;
It would enable high-resolution input control to query input-output proportionality; and
It would exert precise control of recurrent stimulation dynamics to interrogate the relationship between past stresses and future ones.

Turning now to the drawings and data, many embodiments are directed to methods to control the ISR. Numerous embodiments are directed towards optogenetic control to control the ISR. Optogenetic control can be provided by an engineered ISR kinase that can dynamically stimulate the ISR pathway. In many embodiments, controlled light stimulation enables the selective activation of a single optogenetic kinase of the ISR pathway without inducing molecular damage, greatly reducing the combinatorial complexity of the signals that are turned on by chemical, physical, and pathogenic stressors. By controlling the intensity of light stimulation, the fraction of optogenetic proteins the activated state can be controlled, thereby precisely altering the perceived amount of stress. Additionally, quickly toggling lights between on and off positions in a matter of seconds precisely enable the delivery of precisely defined input dynamics. The various embodiments of optogenetic tools are scalable and/or capable of high-throughput assessment, while still allowing for precise dissection of the molecular components of the ISR pathway.

Many embodiments are directed to engineered ISR effectors to be responsive to and activated by light—i.e., optogenetic control. In some embodiments, ISR effectors are defined as a protein that can effect a signal transduction of the ISR. An ISR effector can be an enzyme, a receptor, a kinase, a transcription factor, an initiation factor, a protein chaperone, a regulator of cell cycle or apoptosis, an activator, a repressor, or any other protein that effects response related to cellular stress. Examples of ISR effectors include (but are not limited to) Akt, ATF3, ATF4, ATF6, BBC3, BIM (BCL2L11), CHOP, CK1, CK2, CReP (PPP1R15B), DR5, eIF2α, GADD34 (PPP1R15A), GCN2, HRI, HSP70, HSP90, IRE1, JNK, mTOR, NF-κB, p53, PERK, PI3K, PKA, PKR, PP1, TRB3 (SKIP3), and XBP1. Notably, the list of ISR effectors are human gene products, but it should be understood that ISR effector homologs of other species can be utilized as well.

Several embodiments are directed towards construction of optogenetic ISR effectors. In some embodiments, to construct optogenetic ISR effectors (e.g., opto-PKR), the activating domain(s) of the natural ISR effector are not utilized and photo-switchable variants of Cryptomchrome2 (Cry2) are utilized instead, a light-inducible oligomerizer domain. Accordingly, in some embodiments, an optogenetic ISR effector excludes the activating domain(s) of its cognate natural ISR effector. In some embodiments, the effecting domain, and optionally other domains that are importing in effecting the response, are utilized along with the Cry2 domain(s) to yield an optogenetic ISR effector construct. An activating domain is to mean the natural domain that receives or detects a signal, resulting in the initiation of the effector's activation. The effecting domain is to mean the natural domain that effects the downstream response. For instance, PKR is an innate immunity effector of vertebrates that detects viral and endogenous double-stranded RNA. Accordingly, in one example to yield light responsive PKR (i.e., opto-PKR), PKR's double-stranded RNA binding domains (dRBM1 and dRBM2), which are utilized to detect the presence of dsRNA, can be removed and photo-switchable variants of Cry2 and be combined with the PKR's natural kinase domain. Examples of activating domains include (but are not limited to) stress-sensing domains, nucleic acid sensing domains, transactivation domains, receiver domains, and domains that interacts with upstream biomolecules resulting in effector activation. Examples of effecting domains include (but are not limited to) kinase domains, DNA binding domains, and domains that effects activation or response of a downstream biomolecule.

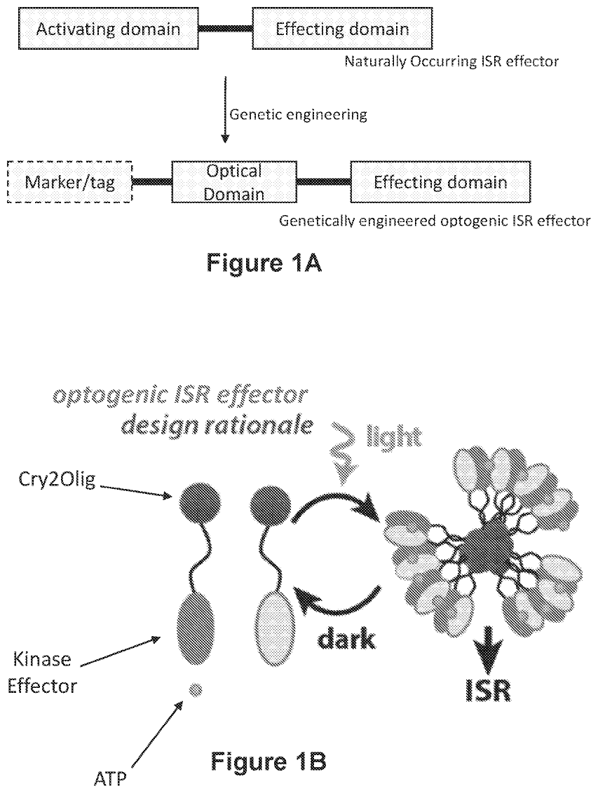

FIG. 1A provides schematic of engineering an optogenetic ISR effector. In this example, a naturally occurring ISR effector comprises an activating domain and effecting domain. A naturally occurring ISR effector may contain other domains as well, such as transmembrane domains, structural domains, enzymatic domains, localization domains, etc., but these domains are omitted in this schematic for simplicity. By means of genetic engineering, an optogenetic ISR effector is generated. In some embodiments, genetically engineered optogenetic ISR effector includes the effecting domain but lacks a functional activating domain of the cognate natural ISR effector, and instead incorporates a functional optical activating domain (e.g., Cry2 domain). In some embodiments, the optogenetic ISR effector lacks an activating domain of the cognate natural ISR effector. In some embodiments, the optogenetic ISR effector includes an activating domain of the cognate natural ISR effector that has been mutated or otherwise altered such that it is not capable of effecting a downstream response. The genetically engineered optogenetic ISR effector may optionally include a marker or tag, such as a fluorescent protein or antigenic tag, which can be useful for visualization, identification, and/or further molecular analysis. Other domains can be included, especially domains of the natural ISR effector that improve or are integral to effecting downstream activation. For example, membrane-bound ISR effector may include a transmembrane domain, a lipid anchor domain, etc. It is noted that there is no requirement for an optogenetic ISR effector to have the same order of domains along the polypeptide as the naturally occurring ISR effector, and the domains can be altered, which may (for example) yield a more effective response and/or enable certain experimental assessments. Furthermore, an optogenetic ISR effector may include domains that are not present in the naturally occurring ISR effector. Generally, and in accordance with several embodiments, an optogenetic ISR effector includes an optical activating domain and an effecting domain that effects a response that is representative of the response provided by effecting domain of the cognate naturally occurring ISR effector. Notably, an optogenetic ISR effector can effect a downstream response in the ISR pathway without external stressors.

FIG. 1B provides a schematic of optogenetic ISR effector. In this example, the optogenetic ISR effector comprises Cry2 oligomerizing domain and a kinase effector. Light energy is impinged upon the optogenetic ISR effector resulting in oligomerization of the Cry2 domains, activating the kinase effector resulting in cross-phosphorylation. The cross-phosphorylation in turn effects the ISR. Removal of the light energy results in de-oligomerization, inactivating the kinase effector such that the optogenetic ISR effector is no longer effecting a response.

Cry2 variants can be selected from one or more of Cry2Olig, Cry2OClust, Cry2ODrop, and/or another variant to enhance protein oligomerization efficiency via light energy. In certain embodiments, the enhanced protein oligomerization efficiency is derived from an E490G mutation in Cry2. Certain embodiments can include C- and/or N-terminal peptide sequences, which increase dynamic exchange with the cytoplasm.

Additional embodiments are directed to nucleic acid constructs comprising a sequence for encoding an optogenetic ISR effector. In some embodiments, a nucleic acid construct can further include additional sequences to enhance and/or control expression of an optogenetic ISR effector, including transcription start sites, a Kozak sequence, polyA signaling site, promoters, enhancers, introns, conditional activators, and/or any other relevant component in the provision of transgene expression. The additional sequences can be operatively linked to the optogenetic ISR effector sequence such that it effects its intended operation upon the expression of the gene. In certain embodiments, a nucleic acid construct may include additional features, such as an origin of replication, one or more splice sites, restriction enzyme cut sites, and other features for propagation, cloning, inserting, or molecularly tagging transgenes. Certain nucleic acids are circular (e.g., plasmid, BAC, etc.), while other nucleic acids are linear. The nucleic acid can be DNA, RNA, DNA/RNA-hybrid, and/or any other applicable nucleic acid molecule for transgene expression. Synthesis and/or manufacturing of either a nucleic acid or a peptide encoding an optogenetic ISR effector can be accomplished through any known or relevant method, including in vivo, in vitro, and chemical synthesis methods.

Further embodiments are directed towards a biological cell comprising an optogenetic ISR effector or a nucleic acid construct encoding an optogenetic ISR effector. Such cells may genetically express an optogenetic ISR effector or may be contacted with an optogenetic ISR effector. In some methodologies, a cell is contacted with a nucleic acid that encodes an optogenetic ISR effector (e.g., via transfection, viral transduction, electroporation, or other delivery means). Any biological cell can be utilized. Such cells can be selected from plant, yeast, fungus, bacteria, animal, mammal, bird, reptile, fungus, human cell, including established cell lines and/or primary cell cultures or tissues. Examples of cell cultures include Hela cells, human osteosarcoma epithelial cells (U2OS), H4 neuroglioma cells (H4), Chinese hamster ovary cells (CHO), HEK-293T cells, and/or any other relevant cell. Nucleic acids encoding an optogenetic ISR effector can be extrachromosomal, episomal, integrated into the native genomic DNA, localized to the cytosol, localized to the nucleus, transiently expressed, constitutively expressed, conditionally expressed, or otherwise in contact with the cell. Optogenetic ISR effector proteins can be localized to the nucleus, localized to the ER/Golgi, localized to the cellular membrane, localized to the cytosol, localized to the extracellular space, localized to mitochondria, localized to stress granules, localized to lysosomes, or otherwise in contact with the cell.

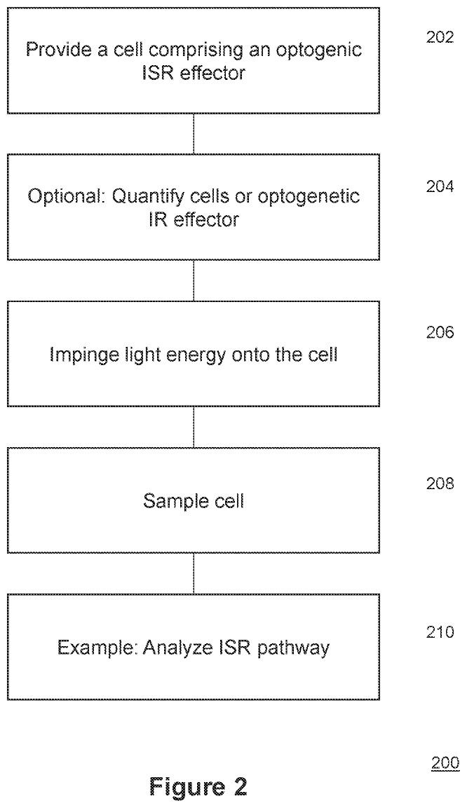

Further embodiments are directed to methods of using an optogenetic ISR effector. For example, FIG. 2 illustrates an exemplary method 200 to use an optogenetic ISR effector. In various processes, this method can be used to assess a cell's ISR, inclusive of assessment of downstream responses. The method can also be used in systems to induce an ISR to yield a particular result.

At 202, many embodiments obtain a cell (or a population of cells) comprising an optogenetic ISR effector. In certain embodiments, a cell culture, a tissue sample, an organ, an organism, an animal comprises the cell (or population of cells) comprising the optogenetic ISR effector. Some embodiments obtain multiple collections of cells, such as in a multi-well plate (e.g., 4-well, 8-well, 16-well, 32-well, 64-well, 96-well, 384-well, 1536-well, etc.), which may be useful in screening assays.

In some embodiments, the cell is contacted with the optogenetic ISR effector. In some embodiments, the cell is contacted with a nucleic acid encoding the optogenetic ISR effector. In some embodiments, the cell expresses the optogenetic ISR effector from a nucleic acid construct encoding the optogenetic ISR effector. The cell (or collection of cells) may proliferate or otherwise expand.

The method may optionally quantify cells at 204. Quantification can be performed to quantify the number of cells comprising an optogenetic ISR effector, the amount of cells expressing an optogenetic ISR effector, the amount of optogenetic ISR effector within a cell, the amount of optogenetic ISR effector expressed in a cell or in a population of cells, which can be performed via various methodologies, such as cell counting, cell sorting, absorbance readings, fluorescence readings (e.g., using a fluorescent tag, fluorescent dye, etc.), flow cytometry, plate readings, cell culture confluency, etc. Other molecular techniques, such as immunodetection techniques (e.g., Western blotting, ELISA, IP, etc.) or nucleic acid detection techniques (e.g., PCR, hybridization, etc.) are utilized to quantify optogenetic ISR effector expression.

Further embodiments impinge light energy onto the cell at 206. An emitter can provide light at a relevant wavelength and intensity for the activation of the optogenetic ISR effector. The light can be UV, IR, and/or visible light, as appropriate for the optical activating domain. The light can be emitted having a selected intensity, a gradient of intensities, a spatial pattern (e.g., using a set of digital micromirrors), a strobing effect (e.g., cycles of illumination), a temporal pattern (e.g., on/off for periods of time), and/or any other relevant emission capability for manipulating activation of the optogenetic ISR effector. In several embodiments, impinging light onto the cell results in activation of the effector domain, effecting a downstream response. In many embodiments, impinging light energy onto the cell is performed without contacting the cell with an external stressor (e.g., chemical, physical, or pathogenic stressor). In many embodiments, impinging light energy onto the cell is performed without external stressors present. In various embodiments, when light is impinged upon the cell, an external stressor is not within or contact with a cell culture, a tissue sample, an organ, an organism, or an animal (as appropriate for the contextual environment of the cell).

At 208, various embodiments sample the cell or collection of cells to assess downstream effects of optogenetic ISR effector activation. Sampling can occur via a single cell or bulk assay. Additionally, sampling can occur at multiple time points or from multiple locations, depending on which factors are of interest. Sampling can include various cellular and molecular assessments such as (for example) biomolecule detection, biomolecule quantification, which can be performed via a number of cellular and molecular techniques. Such techniques can include nucleic acid amplification, nucleic acid hybridization, nucleic acid sequencing, bisulfite sequencing, immunodetection, chromatin immunoprecipitation, fluorescent labeling, protein mass spectrometry, colorimetric assay, microscopy, plate reading, electrophysiology, viability assessments, cytotoxicity assessments, and/or any other relevant cellular and molecular techniques. Such techniques can assess epigenetics, gene regulation, gene transcription, gene translation, protein expression, protein activation, biochemical pathway activation, biomolecule localization, cellular morphology, cell-to-cell interactions, cell motility, etc. RNA sequencing can include bulk sequencing or single cell sequencing to identify an expression pattern as a result activating the ISR effector.

Many embodiments, for example, can analyze pathway effects at 210. For instance, the alterations in protein and/or RNA expression based on spatiotemporal activation of optogenetic ISR effectors can be assessed, especially the effect on various biomolecules within the ISR pathway. Such analysis can be based on qualitative and/or quantitative measures of protein and/or RNA presence within the samples. Further embodiments can characterize resulting biological characteristics, such as by performing gene ontology (GO) analysis. GO analysis can identify families and/or categories of genes that characterize a certain effect. For example, upregulation and/or downregulation of gene or protein expression in the sample can be assessed.

It should be noted that method 200 is merely an example of a method of utilizing an optogenetic ISR effector in accordance with some embodiments. Additional embodiments may add, omit, repeat, or alter certain features without straying from the scope of this disclosure. Certain embodiments may perform certain steps simultaneously and/or in an overlapping manner. Additionally, various embodiments provide optogenetic control of effectors in another pathway.

### Exemplary Embodiments

Although the following embodiments provide details on certain embodiments of the inventions, it should be understood that these are only exemplary in nature, and are not intended to limit the scope of the invention. The examples provided herein highlight the ability to induce an ISR within a cell without having to stress the cell with a chemical, physical, or pathogenic stressor. The examples demonstrate that assessment of the ISR pathway can be achieved without the confounding issues such as cytotoxicity.

### Example 1: Interrogating the ISR Pathway

Background: In this example, PKR is engineered to yield an opto-PKR in which PKR's double-stranded RNA binding domains (dRBM1 and dRBM2) have been removed, and photo-switchable variants of Cry2 have been incorporated. PKR is an innate immunity effector of vertebrates that detects viral and endogenous double stranded RNA, and PKR signaling is dysregulated in numerous diseases, including cancer and neurodegenerative disorders. Optical control of PKR has both a broad significance and specific implications for understanding the fundamental mechanisms cells use to respond to stress. Optogenetic control of PKR has advantages including a high-order self-association, which can be mimicked using optogenetic clustering tools and cytoplasmic localization with sensor domain is isolated to a contiguous stretch of amino acids, simplifying engineering.

While the role of ISR dynamics has not been systematically interrogated, observations suggest it utilizes the modalities of dose and dynamics to encode a wide variety of cell states. PKR-induced ISR activity has been shown to stochastically pulse on the timescales of hours in response to viral infections suggesting host-pathogen modulation of ISR dynamics. More generally, the difference between an adaptive and a maladaptive stress response is commonly defined by the difference in dosage and timing of a stress input (e.g. low, repeated doses of stress induce acclimation whereas larger chronic doses cause physiological dysfunction). Both examples suggest that hysteresis, history-dependent dynamics, in the ISR may play a functional role in dictating outcomes.

To understand how the ISR decodes stress signaling features, intensity and temporal dynamics of opto-PKR activity were systematically controlled. The precise input control methods were coupled with quantitative, time-resolved analysis of global transcriptomic changes as well as single-cell measurements of individual ISR effectors. This strategy revealed transcriptome remodeling governed by two dynamical modes—transient and gradual—that are associated with adaptive and terminal ISR outcomes. By systematically varying virtual stress intensity it was determined that p-eIF2α, the adaptive transcription factor ATF4, and the pro-apoptotic transcription factor CHOP, all respond proportionally, but with fixed temporal phases. These insights were leveraged to develop an ordinary differential equation (ODE) model which quantitatively predicted that both the duration of past stress and the recovery time after stress determine cellular stress memory. Experimental validation of this model mapped the landscape of cellular adaptation to stress.

### Methods

Cell Culture: Human osteosarcoma epithelial cells (U2OS) and H4 neuroglioma cells (H4) were cultured in Dulbecco's Modified Eagle Medium (DMEM) containing 4.5 g/mL glucose (Gibco), 10% fetal bovine serum (Atlas Biologicals, F-0500-D), 1% penicillin and streptomycin at 37 °C and 5% carbon dioxide. All cells carrying light responsive proteins were maintained and treated in the absence of blue light (dark or red light only). Cell culture vessels containing light responsive cells were wrapped in aluminum foil to prevent accidental light leakage. Prior to microscopy experiments, cells were plated onto glass bottom dishes pre-treated with bovine fibronectin to allow for adhesion.

DNA Constructs: The DNA regions of interest were amplified from host plasmids by PCR using CloneAmp HiFi PCR Premix (Takara, 639298) with custom primers for Gibson assembly (with overhangs). Constructs of interest were generated through subcloning into a lentiviral expression vector bearing the SFFV promoter using Gibson Assembly as per the manufacturer's recommendations. The resulting lentiviral expression vectors contained the chimeric PKR constructs. Constructs are available on Addgene.

Lentivirus Transduction: Lentiviral supernatant was produced by transfecting second generation helper constructs, pCMVdR8.91 and pMD2.G (gift of Jared Toettcher, Princeton University), together with our lentiviral constructs at a mass ratio of 0.444:0.056:0.500, respectively, into HEK-293T cells. Transfections were carried out using polyethyleneimine. HEK-293T cells were maintained for 48 hours post transfection at which point the supernatant was harvested, filtered through 0.45 μm filter and stored at −80 prior to infection of target cells.

Fluorescence Activated Cell Sorting: Engineered cells were sorted using Sony SH-800 cell sorter system. Gating was determined using untransduced parental cells as a negative control, and selected cells were sorted through a narrow fluorescence gate to obtain a pseudo-clonal cell line. All engineered cell lines used in this study originated from expansion of pseudo-clonal cell lines.

Western Blotting: Roughly 1×106 cells per experimental condition were harvested using 1× Laemmli buffer (30 mM Tris-HCl pH 6.8, 1% SDS, 10% (w/v) Glycerol, bromophenol blue) from a single well of a 6-well plate immediately after treatment. The lysates were heated at 95 °C for 3 minutes, briefly sonicated, supplemented with 5% 2-mercaptoethanol, and stored at −20 °C conditions until SDS-PAGE. Samples were separated in 10% SDS-PAGE gels run at 100V for 1-2 hours, transferred onto PVDF membranes using a TE 77 semi-dry transfer unit (Cytiva Amersham) at 10V for 15 min. The membranes were blocked with 3% BSA in TBS-T for 1 hour, followed by an overnight incubation with primary antibody at 4 °C. Membranes were washed and incubated with fluorescently or HRP conjugated secondary antibodies for 1 hour at room temperature. All incubation steps were followed by 3× washes with TBS-T for 10 min. The membranes were imaged using enhanced chemiluminescence using a Radiance Plus imager (Azure Biosystems) or a Li-Cor Odyssey instrument. All antibodies were diluted in 3% BSA in TBS-T.

Puromycilation of Nascent Peptides: Cells were grown in culture medium until 80% confluency in 6-well plate for a day (one well per condition). Cells were then treated with activating blue light illumination accordingly until 30 min prior to the end of their treatment, at which point, the culture media was replaced with media containing 18 μM puromycin (Gibco, A1113803) and the cells were incubated for an additional 30 min before sample collection and analysis as described in the Western blotting section.

Immunofluorescence: Cells were grown on fibronectin (Sigma, F1141) coated glass bottom 96 well plates (Cellvis, P96-1.5H-N), washed with PBS, and fixed using 4% paraformaldehyde (PFA, Fisher Scientific, AA433689L) in PBS for 10 minutes at room temperature under dark conditions. Fixed cells were washed with PBS and permeabilized with blocking buffer (0.05% saponin, 0.5% BSA, 50 mM, NH4Cl in PBS) for 30 min at room temperature. The fixed cells were incubated with primary antibodies diluted in blocking buffer overnight at 4 °C. The next day, the cells were washed gently 3× with PBS-T, followed by secondary antibody incubation for 1 hour at room temperature in dark conditions, followed by another round of 3× PBS-T washes. The prepared fixed cells were imaged as detailed in Microscopy imaging.

Microscopy Imaging: All microscopy imaging, live or fixed, was done using a W2 SoRa spinning-disk confocal microscope equipped with an electron-multiplying charge-coupled device camera (Andor, DU-888) and an incubation chamber maintaining cells at 37 °C and 5% CO2. All cells that were imaged were plated on fibronectin (Sigma, F1141) coated glass bottom 96 well plates (Cellvis, P96-1.5H-N). Images were captured using a 40× NA 1.25 water immersion objective and a 60× NA 1.2 water immersion objective.

Optogenetic Stimulation: Engineered photo-switchable proteins were activated using 455 nm blue light. For Western blot experiments with maximal light activation of the pathway, a custom 36 LED plate mounted on a box lined with aluminum foil was utilized, allowing for equal light distribution throughout the box. For confocal imaging, light was delivered through a 488 nm laser at 100% power using a 40× NA 1.25 water immersion objective. Spatial patterning of light was achieved utilizing purpose-built microscope-mounted LED-coupled digital micromirror devices (DMDs) triggered via Nikon NIS Elements software, allowing us to specify the exact parameters of light delivery at each coordinate of our imaging plane. For precisely modulated IF experiments, the benchtop LED array light delivery device (optoPlate) was utilized. The light delivery parameters for the optoPlate were purpose-set for each experiment, where stimulating light was continuously ON or OFF, without pulsing, and 0.2-7.5 mcd range of intensities applied translates as 3-96 on the intensity setting of the optoPlate software. The light illumination sequence of each well included an OFF (dark) period at the start, so that all well illuminations would end at the same time.

Image Analysis: All quantification of images followed the same general flow from creating a TIFF library of images to clean-up and analysis using custom code on MATLAB. Nikon-generated ND2 files were channel separated and converted to TIFF files using ImageJ FIJI macro (ImageJ). The TIFF library of images was organized into separate stacks based on applied treatment (i.e. different intensity of activation, etc.) over time on MATLAB, for ease of access and indexing. All images were then cleaned up to reduce noise through background subtraction and deletion of super bright pixels. DAPI stained nuclei were segmented to identify nuclei as follows: First, the images were Gaussian-blurred, then threshold detection was used to identify edges, and the nuclear mask was watershed transformed in case of connected nuclei. The nuclear masks were quality checked and the threshold was adjusted manually in case of low quality masking. For nuclear localized information, the nuclear mask was applied to the channel of interest and collected information of every nucleus separately. For cytoplasmic information, the nuclear mask was dilated and the mask was removed from the dilated mask to quantify the ring around the nucleus as cytoplasmic information. For condensate information, clusters were identified through both “Laplacian of Gaussian” edge detection and intensity threshold detection and combined the information to create a condensate mask. Non-condensate information was removed from the mask as follows: outside our size threshold and nuclear localized. Mean intensity, location and area of all objects were saved for quantitative analysis on a single cell level for nuclear and cytoplasmic fluorescence normalized to no treatment control. Condensate data was quantified by summing total condensate intensity across an imaging field and normalized to the number of nuclei detected within said image field to obtain the mean condensate fluorescence per cell.

Bulk RNAseq: Sample treatment and extraction: H4 cells carrying Cry2Olig-PKR cells were plated on fibronectin treated 96-well glass-bottom plate. Cells were illuminated according to experimental set-up using the optoPlate device set to continuous illumination intensity of 7.5 mcd as mentioned in Optogenetic stimulation. Cells were immediately lysed using 120 μL Trizol (Gibco, 15596026) per well, under red light, and incubation for lysis for 5 min at room temperature. RNA was extracted according to the manufacturer's protocol.

Illumina library preparations: Bulk RNAseq Illumina libraries were prepared following an adaptation of the single-cell method “CELSeq2” where every illumination duration was treated as a single cell. Briefly, sample RNA was reverse transcribed with Cel-Seq2 primer #4 (25 ng) using Superscript II reverse transcriptase. Second Strand Synthesis (SSS) was carried out via E. coli ligase (Invitrogen, Cat. #18052-019) and DNA polymerase I (Invitrogen, Cat. #18010-025) in SS buffer for 2 hours at 16 °C. Next, cDNA was purified with 1:1 AMPure XP DNA beads (Beckman Coulter, Cat. #A63881), and washed with 80% ethanol twice, before elution into nuclease-free water and vacufuge concentrated. In vitro transcription was then carried out using MEGAscript T7 kit (Thermo Fisher Scientific, AMB13345) for 13 hours at 37 °C, followed by RNA treatment using EXO-SAP (Thermo Fisher Scientific, 78200.200.UL) for 15 min at 37 °C, and finally the samples were fragmented (200 mM Trisacetate (pH 8.1), 500 mM KOAc, 150 mM MgOAc) at 94 °C for 3 min before being quenched by 0.5 M EDTA. The fragmented RNA was size selected with 0.8× AMPure RNA beads (RNAClean XP Kit, Beckman Coulter, Cat. #A63987) and eluted in nuclease-free water.

Bioinformatic analysis: Paired-end sequencing of the libraries was performed on an Illumina NextSeq 500, and hits were quantified and z-score normalized per gene. Transcriptomic data was then analyzed through custom pipeline on python packages “numpy” and “pandas”. As data preparation, all genes that were missing entry at any time were removed from data matrix. SVD function from “numpy” was applied to gene matrix, where rows were distinct genes and columns were duration of stimulation in increasing order, then sorted the data based on U matrix of SVD decomposition. Centroid clustering was also applied on the gene matrix through clustermap function of “seaborn” package.

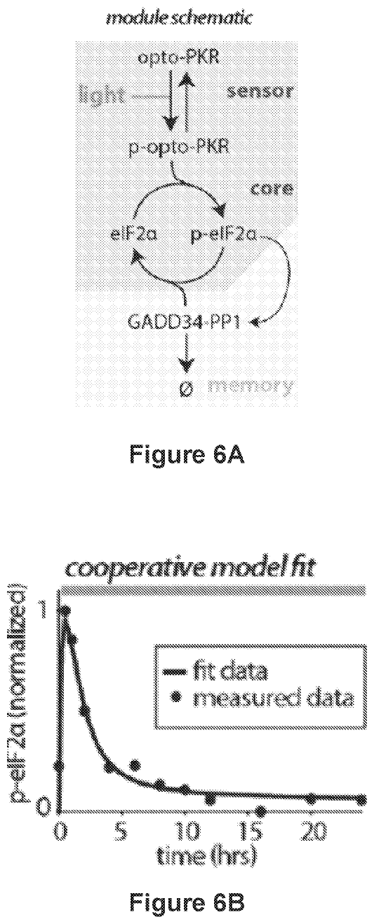

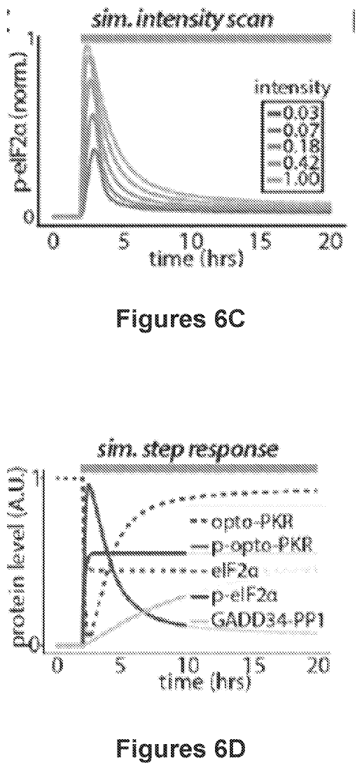

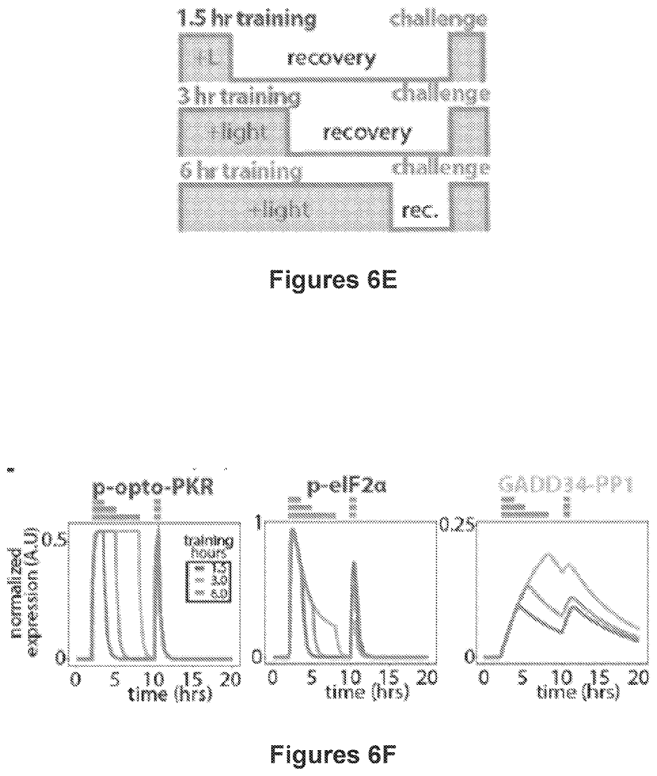

Simulating the mathematical model of ISR: Model simulations were conducted in Python 3 using the Anaconda 4.1.3 using the packages “numpy”, “pandas” and “seaborn”. Numerical simulations were conducted using the “odeint” function from the SciPy v1.8.0 scipy. integrate module. Upon defining the ISR model as ISR_linear and ISR_U functions with all the state variables and parameters, for linear and cooperative models, respectively. Non-linear variable light modulation was defined using Ustep(t), as a range of [0] to [1] as a proxy for illumination at specific intensities. PKR and p-PKR defined in the model refers to the engineered opto-PKR and its phosphorylated form, and Ustep(t) was incorporated into these state variables. Initial conditions of the state variables were set to 0 for phosphorylated proteins (p-PKR, p-eIF2α) and GADD34, while nonphosphorylated proteins (PKR, eIF2α) were set to 1, as shown at time zero of simulated step response (FIG. 6D). All state variables were then plotted using matplotlib plotting library and/or seaborn. heatmap library. For step response simulation, Ustep to [1] was set at time 2 hrs (of modeled time), and the simulation ran through 20 hours. For intensity scan simulation, the magnitude of Ustep was queried from [0.03] to [1], by increasing it from [0] at 2 hrs of modeled time. For the input-recovery ratio simulation, Ustep was pulsed to [1] starting at 2 hours for corresponding length of modeled time (1.5, 3, or 6) hrs, before pulsing for 30 min (0.5 hrs) at time 10 hours. For simulated stress memory landscape, light durations were log sampled (Ustep==1) and recovery durations (Ustep==0), from [0.5] to [10] hours and [2] to [48] hours, respectively, through nested for loop solving the ISR model with odeint for each condition.

Gene ontology analysis: Genes of interest were obtained from the U vector of SVD analysis. The top 500 genes were analyzed for over/under representation in biological processes of Homo Sapiens using GO Enrichment Analysis tool (Panther, “geneontology.org’) (Ashburner et al., 2000; Gene Ontology Consortium, 2021; Mi et al., 2019). The dataset used for this analysis was GO biological processes complete set and performed Fisher's exact test with correction for false discovery rate. All biological processes found significant were collapsed onto its parent families.

### Results

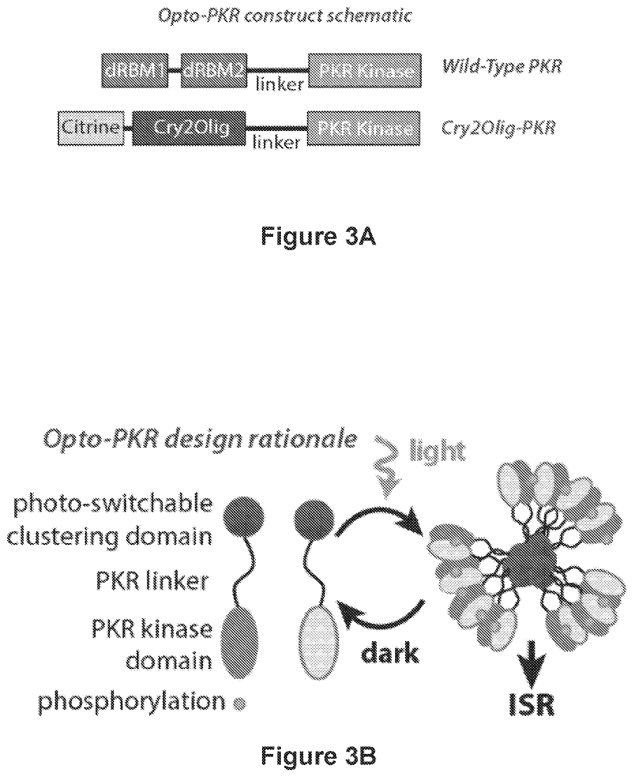

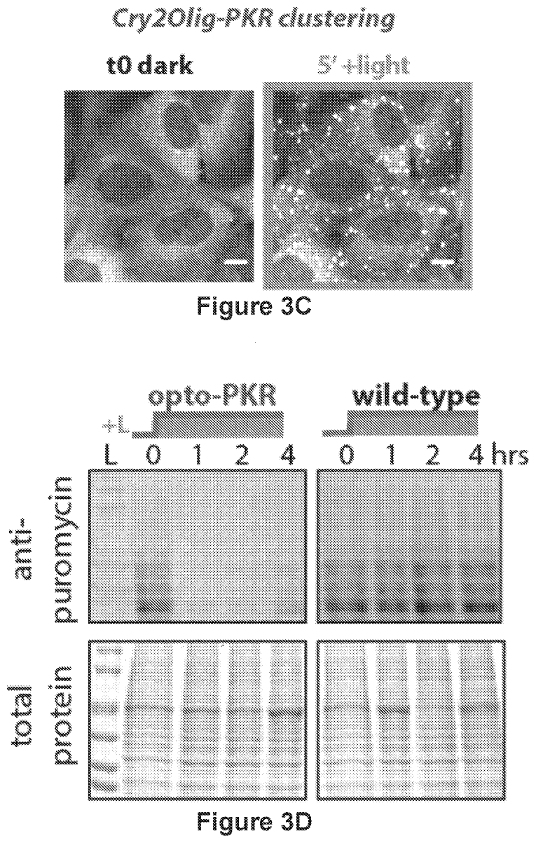

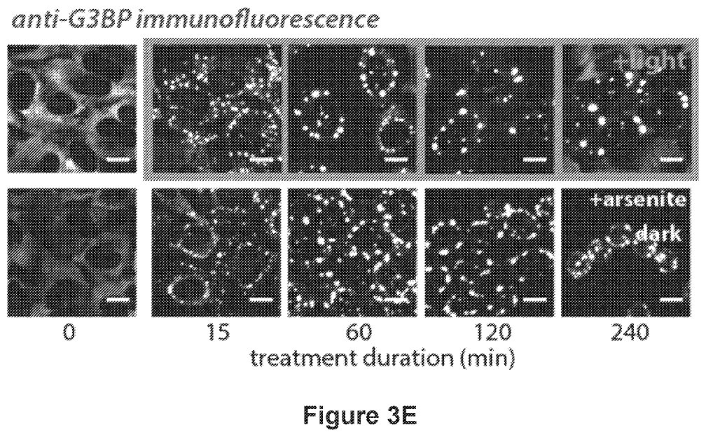

Optogenetic activation of PKR induces the ISR: Optogenetic control over PKR was chosen for two reasons. First, PKR's activation mechanism involves high-order self-association, which can be mimicked using optogenetic clustering tools. Second, PKR is cytoplasmically localized and its sensor domain is isolated to a contiguous stretch of amino acids on the polypeptide chain, making it easier to engineer in comparison to the other ISR kinases. Thus, it was hypothesized that clustering PKR through exchanging PKR's double-stranded RNA binding domains (dRBM1 and dRBM2) with photo-switchable variants of Cryptomchrome2 (Cry2), a light-inducible oligomerizer, would result in light-based activation of the ISR (FIGS. 3A and 3B).

To construct optogenetic PKR (opto-PKR) variants dRBM1 and dRBM2 were replaced with one of three optimized variants of Cry2: Cry2Olig, Cry2OClust, and Cry2ODrop (FIG. 3A). All three variants contain the E490G mutation in Cry2, which enhances the proteins' oligomerization efficiency and is commonly referred to as Cry2Olig. In addition to this mutation, Cry2OClust and Cry2ODrop have C- and N-terminal peptide sequences, respectively, that increase their dynamic exchange with the cytoplasm. In this manner, Cry2Olig-PKR, Cry2OClust-PKR, and Cry2ODrop-PKR sample a range of biophysics, from more solid-like to more liquidlike condensates.

To test if opto-PKR variants formed light-induced clusters H4 neuroglioma cells and U2OS osteosarcoma cells were transduced with each construct, then sorted for expression-matched populations using fluorescence-activated cell sorting and took time-lapse images while continuously stimulating them with activating light. All three opto-PKR variants in both cell lines formed condensates demonstrating light-responsive clustering of the engineered proteins (FIG. 3C). It was noted that the number and size of condensates rapidly increased and then declined for all opto-PKR constructs in both cell lines. An established point mutation (K296R) in the phosphate transfer site of the kinase domain of PKR stabilized these clusters indicating that their activation-induced dissolution requires kinase activity. Next, it was sought to test whether the opto-PKR variants faithfully induce a canonical ISR despite the dynamic changes in cluster size and number. It was first confirmed that none of the opto-PKR variants were constitutively activated in the dark and that illumination of WT H4 cells did not induce phosphorylation of either PKR or eIF2α. The phosphorylation kinetics of engineered PKR (p-PKR, which was resolved on Western blots by its higher molecular weight than endogenous at 130 kDa) and endogenous eIF2α (p-eIF2α) was measured by Western blot in response to continuous illumination. While all opto-PKR variants rapidly induced high autophosphorylation (p-PKR), it was noted that Cry2Olig-PKR maintained higher levels of phosphorylation for the duration of the 72-minute experiment. Cry2OClust-PKR and Cry2ODrop-PKR demonstrated a lower dynamic range and more pulsatile activation kinetics, respectively, at both the level of p-opto-PKR and p-eIF2α. Given its more consistent activation kinetics Cry2Olig-PKR was chose from here on and this variant is referred to as “opto-PKR” in the remainder of these experiments. It was next sought to understand if opto-PKR activation induced canonical ISR-driven translation repression and formation of stress granules (SGs), ribonucleoprotein complexes formed in response to p-eIF2α (Buchan and Parker, 2009). To assess translational repression, opto-PKR was continuously activated and measured puromycin incorporation into nascent peptides using an anti-puromycin antibody. Opto-PKR activation drastically reduced translation initiation measured by nascent peptide puromycilation (FIG. 3D), which is consistent with translational repression observed by ISR activation with chemical inducers. Next, SG formation was monitored by immunofluorescence (IF) of the SG assembly factor G3BP1 upon activation of opto-PKR. Light-induced SGs were indistinguishable from those formed through arsenite induction, except for those observed at late time points (>4 hours) where arsenite treated cells began to detach from the plate and die (FIG. 3E). Altogether the data demonstrate that opto-PKR recapitulates the major ISR events across multiple cell lines, allowing for light-induced control the ISR.

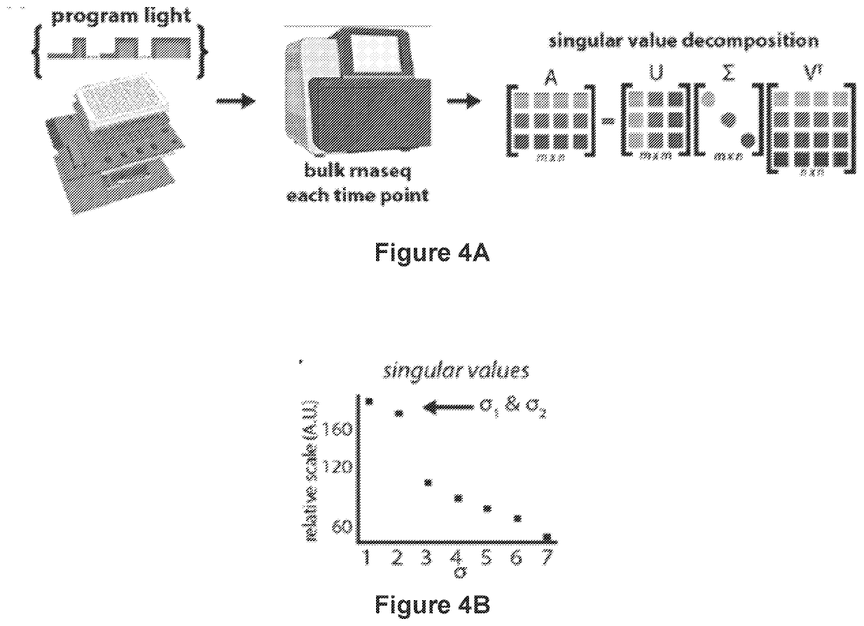

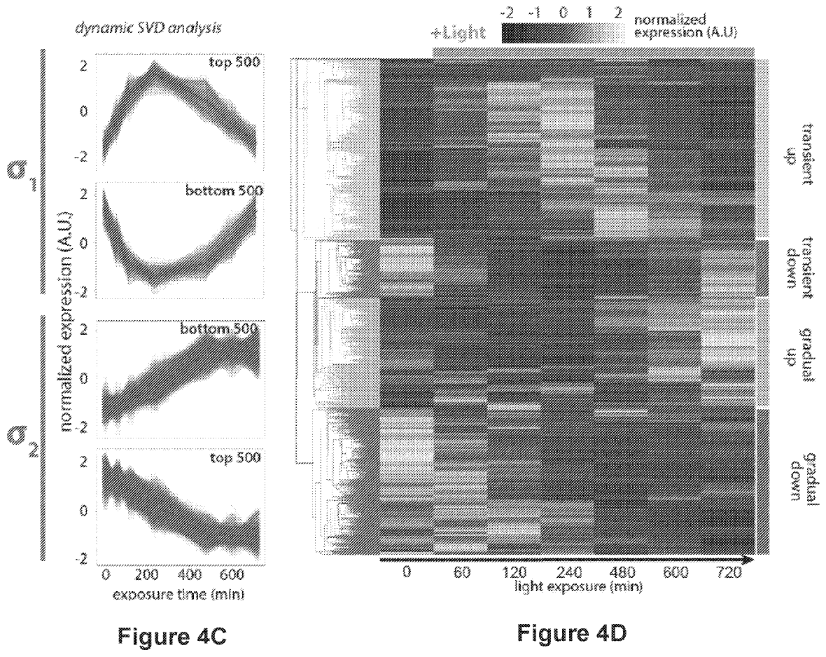

The stress-free ISR has a biphasic transcriptional response: Chemical ISR inducers have pleiotropic effects, rendering them ineffective tools to dissect the cellular responses to stress from cellular damage. To overcome this limitation, opto-PKR was deployed to globally characterize the dynamics of transcriptome remodeling in response to a “stress-free” ISR. It was wondered whether distinct dynamic ISR phases might exist in response to virtual stress. To this end, a time-resolved RNAseq experiment was performed to characterize the dynamic changes in the transcriptome upon continuous stimulation with virtual stress. The optoPlate light delivery device was utilized, which allows for arbitrary control over the illumination sequence of each well of a 96-well plate, to vary the duration of 450 nm light (~7.5 millicandela, see methods for voltages) (FIG. 4A). To obtain time-resolved transcriptional changes, cells were sampled at 7 time points, from 0 to 12 hours. Examination of individual markers of the ISR (e.g. ATF3, DDIT3 (CHOP), PPP1R15A (GADD34), etc.) showed two distinct classes of dynamical responses corresponding with transiently pulsed and gradually accumulating mRNA kinetics.

These two classes of mRNA kinetics suggested that the global transcriptome dynamics may also display a similar pattern. Thus, to map the dynamical gene expression modes of the ISR, a Singular Value Decomposition was performed on the Z-scored, time-resolved transcriptome matrix (FIG. 4A). It was discovered that the first two modes (σ1 and σ2) explained more than half of the total variance of the transcriptome (FIG. 4B). To visualize these two dominant modes, the top 500 and bottom 500 genes from the U vectors associated with σ1 and σ2 were plotted. This analysis showed that σ1 represented a transient response, with genes reaching their maximum between 4-6 hours of continuous activation (FIG. 4C top). On the other hand, genes represented by σ2 showed a gradual, but continuous change that leveled off between 8-10 hours of illumination (FIG. 4C bottom). Analyzing the transcriptome separately through centroid clustering recapitulated these two dynamic modes (FIG. 4D).

Gene ontology (GO) enrichment analysis of these modes hinted that transiently downregulated processes included translation, purine and carbohydrate metabolism, and gonadal mesoderm development, while transiently upregulated processes included protein ubiquitination and TGFb signaling. This analysis suggest that the transient mode is associated with adaptive remodeling of the proteome and alteration of cell identity. On the other hand, gradually upregulated genes were associated with apoptosis and ER and nutrient stress responses, while gradually downregulated processes included mitosis and cell adhesion. This analysis suggests that the gradual mode is associated with a switch towards a terminal response. The regulation of developmental signaling and adhesion by the ISR in neuroglioma cells may reflect oncogenic rewiring. Together, these results indicate that the ISR has two prominent dynamic modes—transient and gradual—and that these modes represent the adaptive and terminal responses of the ISR.

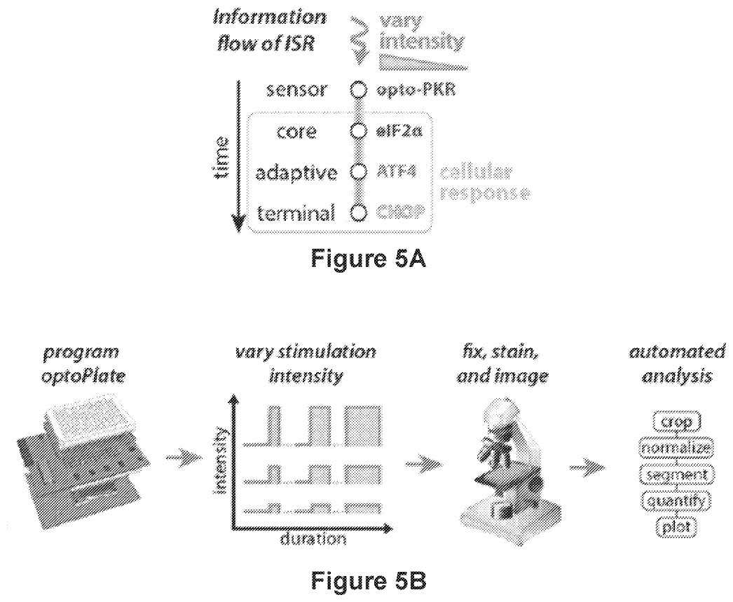

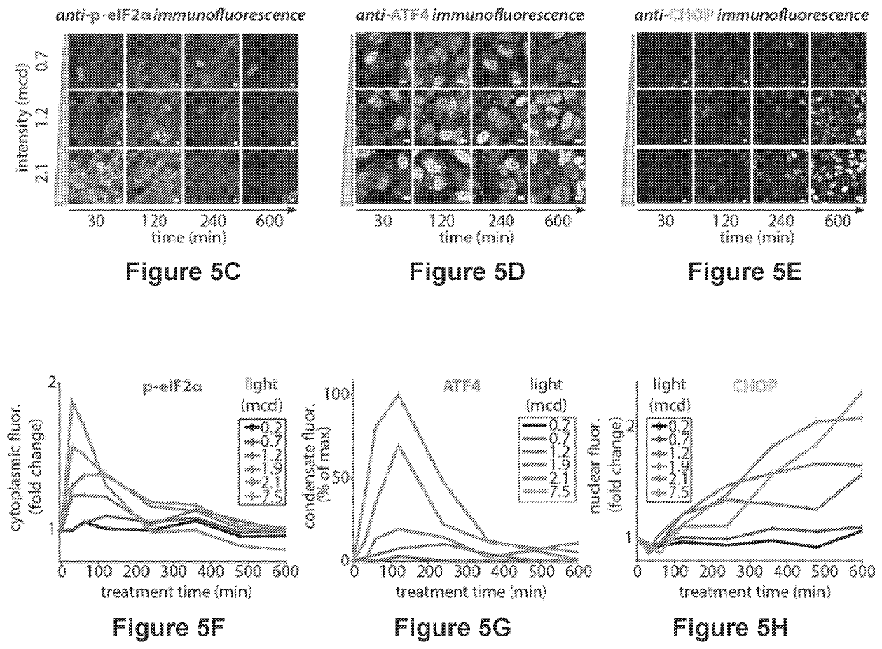

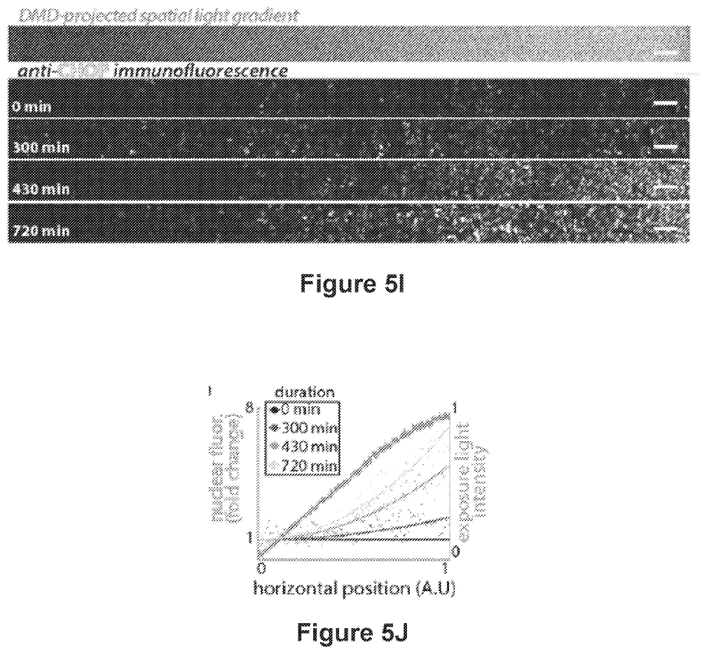

Key ISR transcription factors respond proportionally with fixed phases: The ISR culminates in gene expression programs controlled by adaptive (ATF4) and pro-apoptotic (CHOP) transcription factors, yet the question of how these transcription factors respond to varying the magnitude of a stress has remained unanswered. This question has been difficult to probe with chemical stressors because they induce cascading failures that can lead to a non-linear relationship between chemical stressor concentration and ISR activity. The ISR has been characterized to be switch-like in some instances, while recent findings regarding the mechanism of action of ISR-inhibiting drugs (e.g. ISRIB) suggest a more nuanced dose-response relationship. Thus, it was wondered how the ISR signaling dynamics are affected by varying magnitudes of virtual stress inputs. To this end, the dynamics were characterized at the protein level, of p-eIF2α, ATF4, and CHOP, which represent the ISR core, and its adaptive and terminal phases, respectively (FIG. 5A). To control the magnitude of virtual stress light intensity was varied to control the proportion of Cry2 molecules in the photo-active state. Using the optoPlate device light ranging from 0.2 to 7.5 mcd was applied for over 10 hours and collected time points for IF analysis of p-eIF2α, ATF4, and CHOP to obtain their endogenous dynamic responses (FIG. 5B). To analyze the IF images, a custom image analysis pipeline was developed that extracts single-cell fluorescence intensities from the nucleus, cytoplasm, and SGs, which was used to quantify the response in over 100 cells per condition for a total of more than 10,000 single cells across all times and intensities.

In general, it was discovered that the ISR nodes had features that were modulated in proportion to their inputs, albeit with fixed phases. P-eIF2α peaked within 30 min of activation and then was repressed by 4 hours with the peak magnitude varying in proportion to light intensity (FIGS. 5C-5D). It was found that ATF4 localized to SGs as has been reported previously. SGlocalized ATF4 peaked at 2 hours of activation, where a subset of the adaptive response genes peak, with its peak height also proportional to the input (FIGS. 5E-5F). It was also noted a proportional response in the change in nuclear ATF4, although its magnitude was much lower than SG-localized ATF. Finally, it was found that CHOP accumulated gradually with its rate of accumulation varying with the input intensity (FIGS. 5G-5H).

The proportional intensity encoding of CHOP accumulation was further probed because of its proapoptotic role. A smooth (256 bit) linear gradient of illumination was applied across a field of opto-PKR-bearing cells and IF-stained for CHOP at four time points ranging from 0 to 12 hours (FIG. 5I top). Remarkably, this stimulation led to a spatial gradient of CHOP (FIG. 5I). Quantification of the data showed a best fit corresponding with a 2nd order polynomial, suggesting that the ISR takes the integral of stress inputs when computing CHOP levels (FIG. 5J). Overall, these experiments demonstrate that the peaks of p-eIF2α and ATF4 as well as the slope of CHOP are modulated in proportion to their inputs, while the timing of these responses are independent of input magnitude.

A simple ODE model of the ISR captures core responses and predicts stress memory: The studies so far have focused on probing the cellular response to continuous stress inputs. However, the ISR naturally dynamically oscillates and the proposed adaptive effects of stress are inherently multi-phasic, in that they describe the effect of past stresses on future ones. To predict how dynamic stress inputs are processed, a simple ODE model of the ISR was constructed and fitted. While the ISR has been modeled before, it was sought to build the simplest model that still captured the system dynamics and to be the first to parameterize a model using non-damaging inputs, which could confound the interpretation of the ISR's dynamics.

The ISR was chosen to be represented as three interconnected modules: sensor, core, and memory (FIG. 6A). The sensor module, the ability to activate it with light, and its phosphorylation of eIF2α using mass action kinetics were approximated. To minimize the number of free parameters the core and memory modules were simplified by choosing to represent the GDP and GTP-bound forms of eIF2α, as well as the multiple eIF2B assembly states, as non-linear cooperative interactions between eIF2α, p-eIF2α, and the well-known phosphatase coupling protein GADD34 (FIG. 6A). To parameterize the model, it was fit to the normalized p-eIF2α data collected for the maximal continuous activation shown in FIG. 5D. It was found that the fit, in terms of reduced Chi-Squared statistic, of the simple cooperative model was better than a model lacking the non-linear terms, justifying their inclusion. The model reproduced the negative feedback that drives p-eIF2α back to baseline in response to a continuous step input (FIG. 6B) as well as the proportional modulation of peak p-eIF2α without shifting its phase (FIG. 6C).

Examining the other state variables in response to continuous activation, it was found that the GADD34-PP1 complex accumulated over the course of 20 hrs (FIG. 6d). It was hypothesized that this background accumulation of effective PP1 activity could desensitize future inputs as a function of past inputs. To test this hypothesis, a short (1.5 hrs), intermediate (3 hrs), and long (6 hrs) stimulation was simulated followed by a uniform 30 min challenge, corresponding to the peak activation of the naïve ISR (FIG. 6E). These simulations demonstrated a negative relationship between the amount of prior stimulation on the ability of the core module of the ISR to respond to future challenges, despite the sensor module responding identically for all inputs, which was mediated by the varying accumulation of GADD34-PP1 in the memory module (FIG. 6F). Overall, the model recapitulates the observed ISR dynamics and suggests that the ISR can encode memories of prior stimulations in the GADD34-PP1 feedback control module as has been recently suggested.

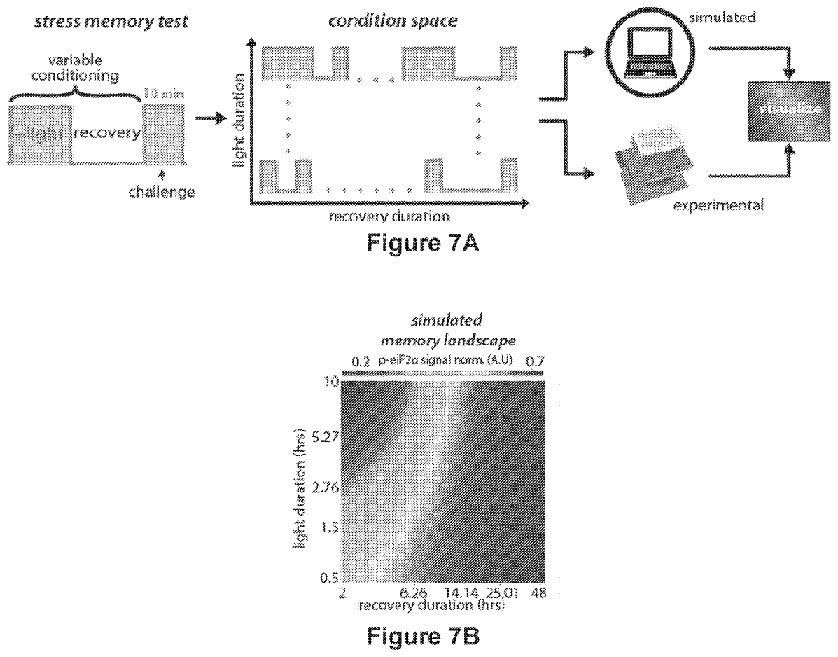

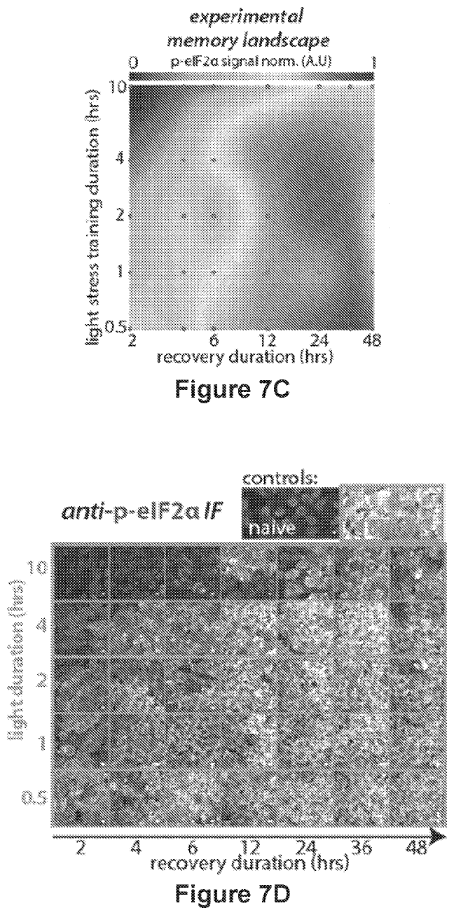

Input dynamics shape the stress memory landscape: Chronically stressed cells respond differently than those exposed to acute stress, suggesting a role for stress input duration in shaping the ISR. The simulations predicted prior inputs attenuate the system's response to future inputs, thus constituting a stress memory. To map the stress memory landscape, the duration of stress input was varied independently from the recovery duration. This stimulation/recovery regime was followed by a uniform challenge (10 min input) to query the cells' response (FIG. 7A). Using this model, these variable training and recovery times were applied to simulate the stress memory landscape. It was found that the p-eIF2α response was dependent on both conditioning variables (FIG. 7B) and anticorrelated with GADD34-PP1 levels at the time of challenge, as expected. Notably, the response of the ISR to the fixed challenge in simulations that were trained with long durations of stress (~3-10 hrs) but given short recovery times (<7 hrs) was severely blunted (FIG. 7B).

To test if this model predicted the stress memory landscape, the optoPlate device was used to probe the response to a subset of the conditioning inputs, followed by the same 10 min challenge. These experiments require rapid reversibility of the input to ensure that the queried cellular state is only a response to the most recent challenge and not to still decaying training inputs. Therefore, the decay of p-opto-PKR was first measured in response to a 20-minute pulse of activating light and found that it decayed on the timescale of 3 mins, an order of magnitude shorter than the briefest recovery duration tested (2 hrs). It was found that cells responded in accordance with the model prediction with long duration inputs and short recovery times severely blunting p-eIF2α levels (FIG. 7C). Short duration inputs also reduced p-eIF2α levels whereas cells were generally fully recovered after >12 hrs (FIG. 7C). Upon examination of IF images, these patterns were visibly evident (FIG. 7D). Finally, the SG marker G3BP1 was costained and found that only after the longest-duration inputs, corresponding to peak terminal phase, did cells completely inhibit SG formation. Together, the simulations and experimental data describe the timescales of cellular adaptation to stress and implicate GADD34 kinetics in determining the memory landscape.

### Conclusions

Neuronal optogenetic tools revolutionized neuroscience because of their ability to causally dissect the role of neural firing dynamics in driving a brain or behavior state. Here the use of cellular optogenetic tools analogously, to control the perceived ISR input and understand how ISR signaling dynamics dictate cellular outcomes. With the ability to deliver arbitrary ISR input dynamics without damaging cells—virtual stress—three longstanding questions were able to be addressed: (i) What are the temporal phases of the ISR? (ii) How do these phases alter the ISR output with varying stress intensities? And (iii) How is the ISR modified by its history?

Based on this analysis of time-resolved transcriptomic profiling, it is proposed that the ISR can be delineated into two distinct dynamical modes: (1) a transient mode associated with the pro-adaptive response that peaks at ~4 hours and (2) a gradual mode that slowly activates terminal response genes in the background, leveling off at ~10 hours. Using this insight, pathway encoding was probed at key nodes, representative of the gradual and transient phases, by coupling a high-throughput light delivery device to custom automated image analysis algorithms. This approach allowed a systematic scan through activation levels and durations in thousands of single cells revealing that they respond proportionally to stress levels, albeit with their own phase-dependent dynamics. Surprisingly, it was found that the timing of these two phases was unchanged by intensity, suggesting that stress duration is a central feature sensed by the ISR.

This interpretation of this data guided construction of a simple ODE model of the ISR to explore its dynamical properties. It was found that the model displays hysteresis, which suggested that it could be used to predict the encoding of stress memories. By systematically modeling variations in the duration of both stress input as well as recovery time, a stress memory landscape was simulated that predicted that longer stresses and shorter recovery times generally blunt the cell's present response. The model's prediction was validated experimentally, generating the first map of the stress memory landscape. Rather than focusing solely on cellular contexts where stress is delivered in a sustained manner using small molecule poisons, this work points to the importance of pulsatile and/or stochastic ISR dynamics that have now been observed to occur in response to viral infection.

The value of ‘virtual’ stress in interrogating pathways that detect molecular damage: Virtually activating the ISR allowed systematic screening through fixed intensities of perceived stress making way for the analysis of a “stress-free” ISR. This allowed examination the global transcriptomic state of cells without confounding alterations due to failing cellular subroutines (such as a damaged ER resulting from thapsigargin treatment, or sublethal oxidation arising from arsenite exposure). By isolating a single sensor, PKR, this approach also allows elimination of any possible contributions of parallel stress response pathways that are activated by the same input, as occurs with multiple innate immunity dsRNA sensors. Interestingly, despite continuously activating the ISR for over 16 hours significant signs of cell death in the cell lines studied were never observed. This could be explained by either that cancer cells have (i) re-wired their stress response to avoid or actively suppress apoptosis, (ii) that in certain cell types ISR-induced programmed cell death requires additional inputs which are satisfied by true damage, or (iii) that cell-death requires longer chronic ISR induction than was tested in this study. Indeed, GO analysis of the terminal phase showed apoptotic processes (GO:0006915) were significantly enriched in the upregulated gene class, indicating a potential preamble to ISR-induced apoptosis. Further, studies of how the ISR is re-wired across a range of cancers using orthogonally-controlled stress sensors could reveal how oncogenic transformation selects for genotypic and epigenetic states that curb ISR-induced cell death.

An additional advantage of optogenetic induction over traditional stressors is that it can be patterned arbitrarily in space through the spatial modulation of light. This property was used to probe the decoding function of CHOP. Using digital micromirror devices with 256-bit resolution we delivered a linear gradient of light to smoothly modulate the ratio of opto-PKR molecules in the photo-active and photoinactive states. Cells produced a 2nd order quadratic response in their CHOP levels, suggesting that cells take the integral of the stress experienced to compute CHOP outputs.

The stress memory landscape in terms of normal cellular functions, adaptation, and stress related diseases: This model and experiments not only make quantitative predictions but also suggest where stress memory is stored and what parameters should be modified to alter the shape of the stress memory landscape. The simulations demonstrate that the instantaneous level of the GADD34-PP1 complex is, at least in part, responsible for the ISR's hysteresis (FIGS. 6E-6F). However, at least another PP1 regulatory subunit CreP also induces dephosphorylation of eIF2α outside the negative feedback loop established by induction of GADD34, and should be accounted for in further evaluation of stress memories. It was found that a cooperative term describing the rate of accumulation of the GADD34-PP1 complex fit the dynamics of p-eIF2α better than a simple mass action term. This suggests that altering the four parameters of this cooperativity function (K, K1, K2, and ß1) as well as the degradation rate of GADD34 (ß2) will enable manipulation of the memory landscape. While the rates of accumulation and decay of GADD34 explain, to some degree, the encoding of stress memories, the possibility that other regulators of the availability of ternary complex (i.e., CreP, eIF2B) could also contribute to the stress memory landscape. Drugs targeting these parameters, for example ISRIB, may prove to be useful in alleviating disease symptoms caused by failure of the cell to either forget or remember past stresses. Further in vivo studies will be required to address this possibility.

In healthy cells challenged with short durations of stress the ISR induces an adaptive response that increases resilience. On the other hand, this same pathway, when chronically activated causes disease marked by reduced cellular function and increased cell death. Since the severity and likelihood of onset of many ISR-related diseases (e.g., Alzheimer's, Huntington's, Parkinson's, and diabetes) increases with age, this observation begs the question of whether the ISR's dynamical information processing capability is prone to corruption during aging. Recently, the role of altered signaling dynamics was shown to explain how some cancers misperceive extracellular signals, transmuting healthy dynamical information into oncogenic signals. Whether altered ISR dynamics explain disease states in a similar manner, with age-related changes in the ISR network altering the healthy cellular perception of stress, remains an open question. Future studies combining precise optogenetic stimuli with live-cell fluorescent reporters of canonical stress responding nodes, such as fluorescent protein CRISPR-tagged ATF4 and CHOP, in models of ISR-associated diseases may illuminate this question.

### Example 2: Opto-IRE1 Construct

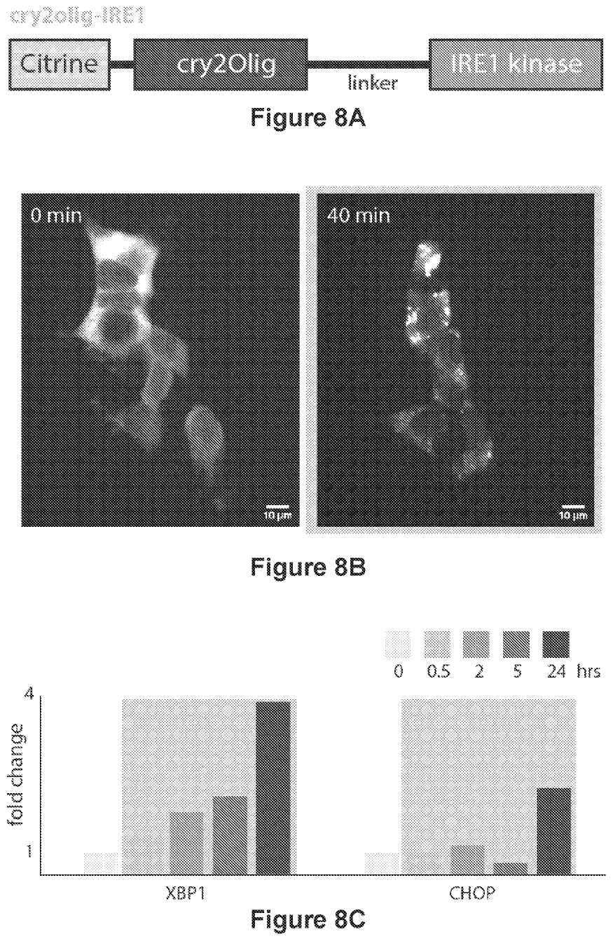

An optogenetic ISR effector based on IRE1 was designed and constructed. Opto-IRE1 is a translational fusion between Cry2Olig and the IRE1 linker and kinase domain, tagged with citrine fluorescent protein on the N-terminal end (FIG. 8A). Cry2Olig domain changes conformation under blue light induction to cluster, leading to IRE1 kinase domain self-association, leading to XBP1 splicing.

The opto-IRE1 construct was stably expressed in HEK293T cells. The opto-IRE1 tool was stimulated via blue light application of 488 nm every 30 seconds for 40 minutes (FIG. 8B right panel). Opto-IRE1 forming clusters in response to light activation show intended function of optogenetic domain of Cry2Olig. Image prior to stimulation showed no puncta clustering (FIG. 8B left panel).

The opto-IRE construct was activated in HEK293T cells via constant 450 nm blue light application for up to 24 hours. Quantitative PCR was performed to assess the expression of XBP1 and CHOP over the period of application (FIG. 8C). XBP1 and CHOP increased over time, suggesting that light-mediated oligomerization of opto-IRE1 recapitulates canonical IRE1 function and the resulting UPR stress response.

### Example 3: Opto-PERK Construct

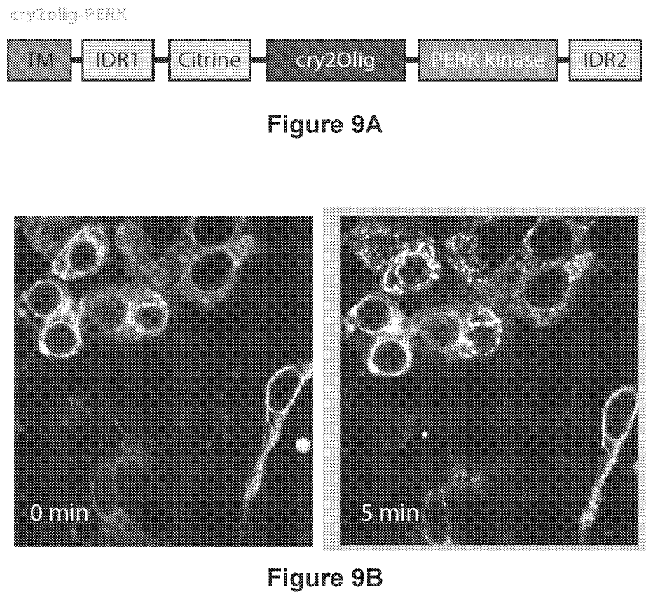

An optogenetic ISR effector based on PERK was designed and constructed. Opto-PERK is a translational fusion between Cry2Olig and the PERK kinase domain, tagged with citrine fluorescent protein on the N-terminal end (FIG. 9A). Transmembrane domain (TM) from endogenous PERK is at the N-terminus to facilitate localization to the ER membrane. Endogenous intrinsically disordered regions (IDR1 and IDR2) from PERK are in the engineered construct to increase self-affinity. Cry2Olig domain changes conformation under blue light induction to cluster, leading to PERK kinase domain activation through self-association, phosphorylating eIF2α and induce unfolded protein stress response pathway.

The opto-PERK construct was stably expressed in HEK293T cells. The opto-PERK tool was stimulated via blue light application of 488 nm every 30 seconds for 5 minutes (FIG. 9B right panel). Opto-PERK forming clusters in response to light activation show intended function of optogenetic domain of Cry2Olig. Image prior to stimulation showed no puncta clustering, but does localize to the ER (FIG. 9B left panel).

### Doctrine of Equivalents

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

1. An engineered protein comprising:
a polypeptide comprising an effecting domain of an integrated stress response effector and an optical activating domain.

2. The engineered protein of claim 1, wherein a naturally occurring polypeptide of the integrated stress response effector comprises an activating domain, wherein the polypeptide comprising the effecting domain and the optical activating domain excludes a functional activation domain of the naturally occurring integrated stress response effector.

3. The engineered protein of claim 1, wherein the optical activating domain is a Cry2 domain contains an E490G mutation.

4. The engineered protein of claim 1, wherein the optical activating domain is a Cry2 domain selected from: Cry2Olig, Cry2OClust, Cry2ODrop.

5. The engineered protein of claim 1, wherein the effecting domain is derived from Akt, ATF3, ATF4, ATF6, BBC3, BIM (BCL2L11), CHOP, CK1, CK2, CReP (PPP1R15B), DR5, eIF2α, GADD34 (PPP1R15A), GCN2, HRI, HSP70, HSP90, IRE1, JNK, mTOR, NF-κB, p53, PERK, PI3K, PKA, PKR, PP1, TRB3 (SKIP3), or XBP1.

6. The engineered protein of claim 5, wherein the effecting domain is a kinase domain derived from PKR.

7. The engineered protein of claim 5, wherein the effecting domain is a kinase domain derived from IRE1.

8. The engineered protein of claim 5, wherein the effecting domain is a kinase domain derived from PERK.

9. The engineered protein of claim 8, wherein the polypeptide further comprises a transmembrane domain and an intrinsically disordered region domain.

10. The engineered protein of claim 1, wherein the polypeptide further comprises a fluorescent protein or an antigenic tag.

11.-12. (canceled)

13. A biological cell comprising the engineered protein of claim 1.

14. (canceled)

15. A method of inducing an integrative stress response, the method comprising:
providing the biological cell of claim 13; and
impinging blue light upon the cell.

16. The method of claim 15 further comprising:
performing a cellular or a molecular assessment on the cell.

17. The method of claim 16, wherein performing a cellular or a molecular assessment on the cell comprises imaging the cell via microscopy to assess whether the engineered protein was activated.

18. The method of claim 16, wherein performing a cellular or a molecular assessment on the cell comprises assessing the effect on the integrative stress response pathway upon impinging blue light upon cell.

19. The method of claim 15 further comprising:
delivering a nucleic acid molecule to the biological cell to induce expression of the engineered protein within the biological cell, wherein the nucleic acid molecule comprises:
a sequence encoding the engineered protein, and
a promoter operatively linked to the sequence encoding the engineered protein.

20. The method of claim 19, wherein delivering the nucleic acid molecule comprises one or more of: transfecting the nucleic acid into the biological cell, virally transducing the nucleic acid into the biological cell, or electroporating the nucleic acid into the biological cell.
